(19) United States
(12) Patent Application Publication
(10) Pub. No.: US 2014/0050765 A1
(43) Pub. Date: Feb. 20, 2014

(54) Devices and Methods for Tissue Engineering

(71) Applicant: James Jenq Liu, Mason, OH (US)

(72) Inventor: James Jenq Liu, Mason, OH (US)

(73) Assignee: BIO2 TECHNOLOGIES, INC., Woburn, MA (US)

(21) Appl. No.: 13/585,325

(22) Filed: Aug. 14, 2012

(51) Int. Cl. A61K 33/42; A61P 19/08; A61K 9/00

(52) U.S. Cl. 424/400; 424/602

(57) ABSTRACT

A resorbable tissue scaffold in a glass ceramic composition forms a rigid three-dimensional porous matrix having a bioactive composition. Porosity in the form of interconnected pore space is provided by the space between the glass-ceramic fiber forming the porous matrix. Strength of the porous matrix is provided by the interconnected and bonded fiber that fuses into the rigid three-dimensional matrix. The resorbable tissue scaffold supports tissue ingrowth when implanted as a device for the repair of damaged and/or diseased bone tissue.

### FIELD OF THE INVENTION

The present invention relates generally to osteostimulative medical implants. More specifically, the invention relates to a bioactive implant having osteostimulative properties to promote tissue ingrowth when implanted in living tissue.

### BACKGROUND OF THE INVENTION

Medical devices and implants are used to repair defects in bone tissue in surgical and orthopedic procedures. These devices are used for the replacement or repair of deteriorated bone tissue to enhance or facilitate the body's inherent healing mechanisms to produce rapid healing of musculoskeletal injuries resulting from severe trauma or degenerative disease.

Biocompatible devices and implants that require load-bearing strength have been traditionally made of metallic alloys. While these materials are fully compatible when implanted in living tissue the mechanical properties of these materials do not closely match that of surrounding natural bone that can result in loosening of fixation. Additionally, metallic devices are biopersistent and remain intact as implanted unless removed in subsequent procedures. Biocompatible devices and implants have been developed using materials that are biologically active, including bioactive glass, glass-ceramics, ceramics, and polymers. The advantage of bioactive materials is these materials are bioresorbable and are dissolved and resorbed by the body as the tissue surrounding the implant remodels into bone tissue. These bioactive materials typically do not exhibit the load bearing strength of metallic devices and thus, the application of such devices can be limited.

### BRIEF SUMMARY OF THE INVENTION

The present invention meets the objectives of a bioresorbable medical device and implant for the repair of bone defects by providing a material that is bioresorbable, osteostimulative, and load bearing. The present invention provides a bioresorbable (i.e., resorbable) tissue scaffold of bioactive glass-ceramic fiber composition having an osteostimulative porous structure. The porous structure has interconnected pore space with a pore size distribution in the range of about 10 μm to about 600 μm with porosity between 20% and 85% to provide osteoconductivity once implanted in bone tissue. Embodiments of the present invention include pore space in the range of about 40 μm and about 600 μm.

The tissue scaffold of the present invention is a porous rigid matrix of having a glass-ceramic composition of a bioactive glass phase and a calcium silicate ceramic phase. The rigid matrix is porous having a porosity of about 20% to about 85% due to interconnected pore space within and throughout the porous rigid matrix, the pore space having a pore size distribution sufficient to promote tissue ingrowth when implanted in living tissue. The tissue scaffold of the present invention is formed from amorphous bioactive glass fiber heated in a controlled reaction process to precipitate a ceramic phase in a bioactive glass phase to provide a resorbable and bioactive glass-ceramic composition.

The glass-ceramic composition of the present invention can have a bioactive glass phase with wollastonite as the ceramic phase to provide high strength while retaining bioactive and resorbable characteristics. In an embodiment essentially all of the calcium oxide of the bioactive glass fiber composition is consumed in the formation of the calcium silicate or wollastonite ceramic phase. In an embodiment the ceramic phase comprises greater than 10% (mol) of the glass-ceramic composition, thereby exhibiting significantly greater strength than the amorphous glass fiber of the raw materials used to fabricate the tissue scaffold of the present invention.

The physical properties of embodiments of the present invention include compressive strength of greater than 50 MPa and pore size distribution ranging from about 40 μm to about 600 μm.

### BRIEF DESCRIPTION OF THE SEVERAL VIEWS OF THE DRAWINGS

The foregoing and other objects, features, and advantages of the invention will be apparent from the following detailed description of the several embodiments of the invention as illustrated in the accompanying drawings in which like reference characters refer to the same parts throughout the different views. The drawings are not necessarily to scale, with emphasis instead being placed upon illustrating the principles of the invention.

FIG. 1 is an image representation of an embodiment of the present invention at approximately 100× magnification.

FIG. 2 shows the graphical results of an X-ray Diffraction (XRD) analysis of an illustrative embodiment of the invention.

FIG. 3 is a flowchart depicting a process of fabricating an embodiment of the present invention.

FIG. 4 is a side elevation view of a bioactive tissue scaffold according to the present invention manufactured into a spinal implant.

FIG. 5 is a side perspective view of a portion of a spine having the spinal implant of FIG. 4 implanted in the intervertebral space.

FIG. 6 is a schematic drawing showing an isometric view of a bioactive tissue scaffold according to the present invention manufactured into an osteotomy wedge.

FIG. 7 is a schematic drawing showing an exploded view of the osteotomy wedge of FIG. 6 operable to be inserted into an osteotomy opening in a bone.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

The present invention provides a bioresorbable medical device and implant for the repair of bone defects that is bioresorbable, osteostimulative, and exhibiting load bearing strength upon implantation. The present invention provides a bioresorbable (i.e., resorbable) tissue scaffold of bioactive glass-ceramic fiber composition having an osteostimulative porous structure. The porous structure has interconnected pore space with a pore size distribution in the range of about 10 μm to about 600 μm with porosity between 20% and 85% to provide osteoconductivity once implanted in bone tissue. Embodiments of the present invention include pore space in the range of about 40 μm and about 600 μm.

Various types of synthetic implants have been developed for tissue engineering applications in an attempt to provide an implantable device that mimics the properties of natural bone tissue and promotes healing and repair of tissue. Metallic and bio-persistent structures have been developed to provide high strength in a porous structure that promotes the growth of new tissue. These materials however, are not bioresorbable and must either be removed in subsequent surgical procedures or left inside the body for the life of the patient. A disadvantage of bio-persistent metallic and biocompatible implants is that the high load bearing capability does not transfer to regenerated tissue surrounding the implant. When hard tissue is formed, stress loading results in a stronger tissue but the metallic implant shields the newly formed bone from receiving this stress. Stress shielding of bone tissue therefore results in weak bone tissue which can actually be resorbed by the body, which is an initiator of fixation loosening.

Implants into living tissue evoke a biological response dependent upon a number of factors including the composition of the implant. Biologically inactive materials are commonly encapsulated with fibrous tissue to isolate the implant from the host. Metals and most polymers produce this interfacial response, as do nearly inert ceramics, such as alumina or zirconia. Biologically active materials or bioactive materials, elicit a biological response that can produce an interfacial bond securing the implant material to the living tissue, much like the interface that is formed when natural tissue repairs itself. This interfacial bonding can lead to an interface that stabilizes the scaffold or implant in the bony bed and provide stress transfer from the scaffold across the bonded interface into the bone tissue. When loads are applied to the repair, the bone tissue including the regenerated bone tissue is stressed, thus limiting bone tissue resorption due to stress shielding. A bioresorbable material can elicit the same response as a bioactive material, but can also exhibit complete chemical degradation by body fluid.

The challenge in developing a resorbable tissue scaffold using biologically active and resorbable materials is to attain load bearing strength with porosity sufficient to promote the growth of bone tissue. Conventional bioactive bioglass and bioceramic materials in a porous form are not known to be inherently strong enough to provide load bearing strength as a synthetic prosthesis or implant. Conventional bioactive materials prepared into a tissue scaffold with sufficient porosity to be osteostimulative have not exhibited load bearing strength. Similarly, conventional bioactive materials in a form that provides sufficient strength do not exhibit a pore structure that can be considered to be osteostimulative.

The present invention relates to a glass-ceramic composition in a porous structure that is formed from a controlled crystallization of amorphous bioactive glass fibers. The composition provides high strength while retaining the bioactive characteristics of the bioactive glass raw materials in a porous structure that facilitates tissue ingrowth.

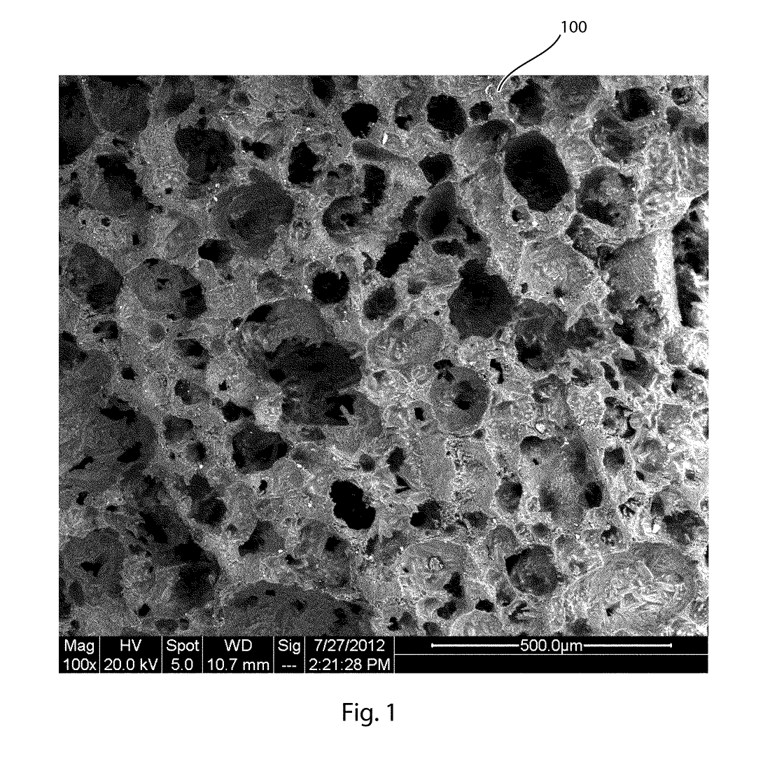

FIG. 1 is an image representation of an embodiment of the present invention at approximately 100× magnification. The porous structure 100 is a rigid three-dimensional matrix having a glass ceramic composition with an osteoconductive interconnected porosity. As used herein, the term “rigid” means the structure does not significantly yield upon the application of stress unit it fractures in the same way that natural bone would be considered to be a rigid structure. As used herein, the term “osteoconductive” means that the material can facilitate the ingrowth of bone tissue. Cancellous bone of a typical human has a compressive crush strength in the range of about 4 to 12 MPa with an elastic modulus ranging between about 0.1 to about 0.5 GPa.

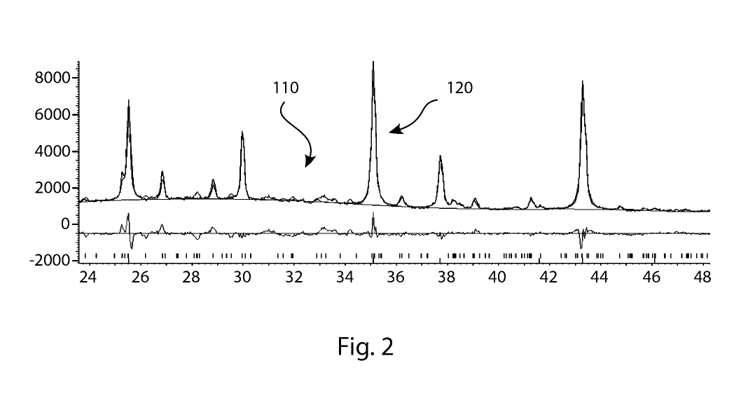

The porous structure 100 of the present invention is a glass-ceramic that has an amorphous phase and one crystalline phases. FIG. 2 depicts an X-ray Diffraction (XRD) analysis of an illustrative embodiment of the invention that shows the presence of an amorphous phase 110 and a crystal phase 120. In the illustrative embodiment fabricated from amorphous silica calcium magnesium sodium potassium phosphate 13-93 bioactive glass fiber the crystal phase composition is indicated to be wollastonite. Wollastonite is a calcium silicate (CaSiO₃) that is formed through a controlled crystallization of amorphous glass fiber in a porous structure.

Glass-ceramic materials exhibit properties of increased strength, toughness, thermal stability, machinability over amorphous glass materials. A glass-ceramic biomaterial can be designed to exhibit specific rates of dissolution and resorption in vivo. For example, wollastonite in a bioactive glass matrix as a glass-ceramic composition can exhibit bioactivity and bioresorption when implanted in living tissue. The present invention relates to the formation of glass-ceramic compositions while retaining a porous, osteostimulative microstructure.

The porous structure 100 of the present invention is fabricated using fibers as a raw material that create a bioactive glass-ceramic composition that has a substantially uniform glass-ceramic composition throughout the porous structure. In this way a bioactive and resorbable device can be fabricated with increased strength and durability provided by the ceramic phase of the material. The fibers can be composed of a material that is a precursor to the glass-ceramic material, and in an embodiment the fiber is a silica calcium phosphate glass. The term “fiber” as used herein is meant to describe a filament or elongated member in a continuous or discontinuous form having an aspect ratio greater than one, and formed from a fiber-forming process such as drawn, spun, blow, or other similar process typically used in the formation of fibrous materials or other high aspect-ratio materials.

Bioactive materials, such as silica- or phosphate-based glass materials with certain compositional modifiers that result in bioactivity, including but not limited to modifiers such as oxides of magnesium, sodium, potassium, calcium, phosphorus, and boron exhibit a narrow working range because the modifiers effectively reduce the devitrification temperature of the bioactive material. The working range of a glass material is typically known to be the range of temperatures at which the material softens such that it can be readily formed. In a glass fiber forming process, the glass material in a billet or frit form is typically heated to a temperature in the working range upon which the glass material is molten and can be drawn or blown into a continuous or discontinuous fiber. The working range of bioactive glass materials is inherently narrow since the devitrification temperature of the glass material is either extremely close or within the working range of the material. In other words, in a typical process for the formation of fiber-based bioactive glass compositions, the temperature at which a fiber can be drawn, blown, or otherwise formed, is above the glass transition temperature but less than the devitrification temperature of the bioactive glass composition. When certain bioactive glass materials are drawn or blown into a fiber form at or near the devitrification temperature, the molten or softened glass undergoes a phase change through crystallization that inhibits the continuous formation of fiber.

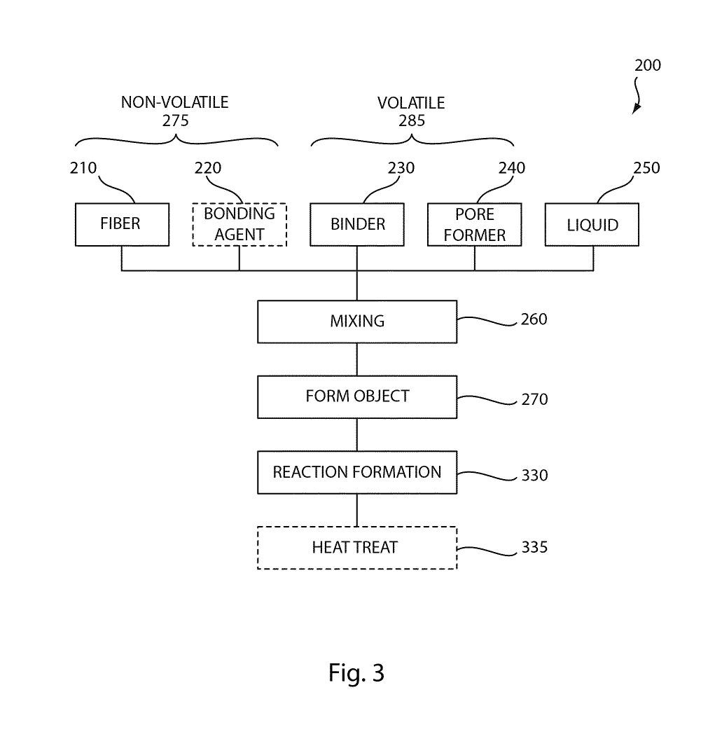

FIG. 3 depicts the method 200 of the present invention to fabricate the porous structure 100 of a bioactive glass-ceramic composition. Generally, amorphous glass fiber 210 is mixed with a binder 230, a pore former 240, and a liquid 250 to form a plastically moldable material which is then reaction formed into a glass-ceramic composition to form the porous structure. A subsequent heat treating process is provided to continue the controlled crystallization of the porous structure into the desired glass-ceramic composition.

The fibers 210 can be provided in bulk form, or as chopped fibers in a composition that is a bioactive glass material. A fiber 210 that is a bioactive glass material includes a fiber having a composition that is at least the components of the desired bioactive glass-ceramic composition. For example, the fiber 210 can be a silica calcium phosphate fiber, or it can be a silica calcium fiber, or a combination of any of the compositions used to form the desired bioactive glass-ceramic composition. The diameter of the fiber 210 can range from about 1 to about 200 μm and typically between about 5 to about 100 μm. Fibers 210 of this type can be produced with a relatively narrow and controlled distribution of fiber diameters or, depending upon the method used to fabricate the fiber, a relatively broad distribution of fiber diameters can be produced. Fibers 210 of a given diameter may be used or a mixture of fibers having a range of fiber diameters can be used. The diameter of the fibers 210 will influence the resulting pore size, pore size distribution, strength, and elastic modulus of the porous structure 100, that will influence not only the osteoconductivity of the porous structure 100, but also the rate at which the porous structure is dissolved by body fluids when implanted in living tissue and the resulting strength characteristics, including compressive strength and elastic modulus.

The fibers 210 used according to the present invention as herein described are typically continuous and/or chopped glass fiber. As described herein above certain bioactive glass compositions are difficult to form as a fiber because the working range of the material is extremely narrow. Silica glass in various compositions can be readily drawn into continuous or discontinuous fiber but the addition of calcium oxide and/or phosphate compounds necessary to create a silica-based bioactive composition are the very compounds that result in the reduction of the working range of the silica-based glass. The use of a fiber 210 that has a composition that is a precursor to the desired bioactive glass-ceramic composition provides for a readily-obtained and easily formed fiber material to form a porous fiber-based structure that is crystallized into the desired bioactive composition during the formation of the tissue scaffold.

Examples of fiber 210 that can be used according to the present invention include silica-based glass fiber. Silica-based materials having a calcium oxide content less than 30% by weight can be typically drawn or blown into fiber form. Silica-based glass materials are generally required to have an alumina content less than 2% by weight since any amount of alumina in excess of that amount will reduce the bioactive characteristics of the resulting structure. Phosphate glasses are precursors to bioactive compositions and can be readily provided in fiber form. These precursor materials that exhibit a sufficient working range can be made into a fiber form through melting in any one of various methods. An exemplary method involves a combination of centrifugal spinning and gaseous attenuation. A glass stream of the appropriate viscosity flows continuously from a furnace onto a spinner plate rotating at thousands of revolutions per minute. Centrifugal forces project the glass outward to the spinner walls containing thousands of holes. Glass passes through the holes, again driven by centrifugal force, and is attenuated by a blast of heated gas before being collected. In another exemplary method, glass in a molten state is heated in a vessel perforated by one or more holes of a given diameter. The molten glass flows and is drawn through these holes, forming individual fibers. The fibers are merged into strands and collected on a mandrel.

Alternative fiber and fiber-like materials that are precursors to bioactive glass-ceramic compositions can be used. For example, a sol-gel fiber drawing method pulls or extrudes a sol-gel solution of the precursor with the appropriate viscosity into a fiber strand that is subsequently heat treated to bind the material into a cohesive fiber. The sol-gel fiber can be formed from a precursor material or a combination of one or more precursor materials that react with each other to create the desired bioactive glass-ceramic composition at the reaction formation 330 step, as described in further detail below. Yet other alternative methods can be used to provide a fiber 210. For example, whiskers and fiber-like segments of silica-based oxides of magnesium, sodium, potassium, calcium, and phosphorus can be provided as the fiber 210.

The binder 230 and the liquid 250, when mixed with the fiber 210, and pore former 240 create a plastically formable batch mixture that enables the fibers 210 to be evenly distributed throughout the batch, while providing green strength to permit the batch material to be formed into the desired shape in the subsequent forming step 270. An organic binder material can be used as the binder 230, such as methylcellulose, hydroxypropyl methylcellulose (HPMC), ethylcellulose and combinations thereof. The binder 230 can include materials such as polyethylene, polypropylene, polybutene, polystyrene, polyvinyl acetate, polyester, isotactic polypropylene, atactic polypropylene, polysulphone, polyacetal polymers, polymethyl methacrylate, fumaron-indane copolymer, ethylene vinyl acetate copolymer, styrene-butadiene copolymer, acryl rubber, polyvinyl butyral, inomer resin, epoxy resin, nylon, phenol formaldehyde, phenol furfural, paraffin wax, wax emulsions, microcrystalline wax, celluloses, dextrines, chlorinated hydrocarbons, refined alginates, starches, gelatins, lignins, rubbers, acrylics, bitumens, casein, gums, albumins, proteins, glycols, hydroxyethyl cellulose, sodium carboxymethyl cellulose, polyvinyl alcohol, polyvinyl pyrrolidone, polyethylene oxide, polyacrylamides, polyethylerimine, agar, agarose, molasses, dextrines, starch, lignosulfonates, lignin liquor, sodium alginate, gum arabic, xanthan gum, gum tragacanth, gum karaya, locust bean gum, irish moss, scleroglucan, acrylics, and cationic galactomanan, or combinations thereof. Although several binders 230 are listed above, it will be appreciated that other binders may be used. The binder 230 provides the desired rheology and cohesive strength of the plastic batch material in order to form a desired object and maintaining the relative position of the fibers 210 in the mixture while the object is formed, while remaining inert with respect to the bioactive materials. The physical properties of the binder 230 will influence the pore size and pore size distribution of the pore space 120 of the scaffold 100. Preferably, the binder 230 is capable of thermal disintegration, or selective dissolution, without impacting the chemical composition of the bioactive components, including the fiber 210.

The liquid 250 is added as needed to attain a desired rheology in the plastic batch material suitable for forming the plastic batch material into the desired object in the subsequent forming step 270. Water is typically used though solvents of various types can be utilized. Rheological measurements can be made during the mixing step 260 to evaluate the plasticity and cohesive strength of the mixture prior to the forming step 270.

Pore formers 240 are included in the mixture to define and enhance the pore space of the porous structure 100 and to provide uniform heating throughout the object during the reaction formation step 330, as described in more detail below. Generally, pore formers are non-reactive materials that occupy volume between the fibers in the plastic batch material during the mixing step 260 and the forming step 270. The particle size and size distribution of the pore former 240 influences the resulting pore size and pore size distribution of the pore space in the porous structure 100.

Particle sizes can typically range between about 25 μm or less to about 450 μm or more, or alternatively, the particle size for the pore former can be a function of the fibers 210 diameter ranging from about 0.1 to about 100 times the diameter of the fibers 210. The pore former 240 must be readily removable during the reaction forming step 330 without significantly disrupting the relative position of the surrounding fibers 210. In an embodiment of the invention, the pore former 240 can be removed via pyrolysis or thermal degradation, or volatilization at elevated temperatures during the reaction forming step 330. For example, graphite or carbon particles can be included in the mixture as the pore former 240. Removal of the pore former during the reaction forming step 330 provides additional heat as the pore former 240 is exothermically oxidized or combusted so that a controlled heating of the glass fiber can facilitate the formation of the desired glass-ceramic composition and thus, the selection of the composition of at least a portion of the pore former 240 may be limited to those materials that provide the requisite temperature differential during heating, as described in further detail below. One skilled in the art would appreciate the availability of alternate material compositions for use as the pore former 240. Additionally, mixtures of a plurality of materials and material compositions can be selected as a pore former 240. Alternative materials include, without limitation: carbon black, activated carbon, graphite flakes, synthetic graphite, wood flour, modified starch, celluloses, coconut shell husks, latex spheres, bird seeds, saw dust, pyrolyzable polymers, poly (alkyl methacrylate), polymethyl methacrylate, polyethyl methacrylate, poly n-butyl methacrylate, polyethers, poly tetrahydrofuran, poly (1,3-dioxolane), poly (alkalene oxides), polyethylene oxide, polypropylene oxide, methacrylate copolymers, polyisobutylene, polytrimethylene carbonate, polyethylene oxalate, polybeta-propiolactone, polydelta-valerolactone, polyethylene carbonate, polypropylene carbonate, vinyl toluene/alpha-methylstyrene copolymer, styrene/alpha-methyl styrene copolymers, and olefin-sulfur dioxide copolymers. Pore formers 240 may be generally defined as organic or inorganic, with the organic typically burning off at a lower temperature than the inorganic. Although several pore formers 240 are listed above, it will be appreciated that other pore formers 240 may be used. Pore formers 240 can be, though need not be, fully biocompatible since they are removed from the porous structure 100 during processing.

Optionally, additional precursors to the desired bioactive glass-ceramic material can be provided as a bonding agent 220 to combine with the composition of the fiber 210 to modify the composition of the glass and/or ceramic phase of the resulting material. The bonding agent 220 can include powder-based material of the same composition as the bulk fiber 210, or it can include powder-based material of a different composition. In an embodiment of the invention the bonding agent 220 can be coated on the fibers 210 as a sizing or coating. In this embodiment, additional precursors to the bioactive composition are added to the fiber, for example, as a sizing or coating. In an alternate embodiment, the bonding agent 220 is a sizing or coating that is added to the fiber during or prior to the mixing step 260. The bonding agent 220 can be solids dissolved in a solvent or liquid that are deposited on the fiber and/or other bonding agent 220 precursors when the liquid or solvent is removed. The relative quantities of the fiber 210 and the bonding agent 220 combined with the heat treatment generally determine the compositions of the respective phases of glass and ceramic components of the porous structure 100.

The relative quantities of the respective materials, including the bulk fiber 210, the binder 230, and the liquid 250 depend on the overall porosity desired in the porous structure 100. For example, to provide a structure 100 having approximately 60% porosity, nonvolatile components 275, such as the fiber 210, would amount to approximately 40% of the mixture by volume. The relative quantity of volatile components 285, such as the binder 230 and the liquid 250 would amount to approximately 60% of the mixture by volume, with the relative quantity of binder to liquid determined by the desired rheology of the mixture. Furthermore, to produce a structure 100 having porosity enhance by the pore former 240, the amount of the volatile components 285 is adjusted to include the volatile pore former 240. Similarly, to produce a structure having strength enhanced by the bonding agent 220, the amount of the nonvolatile components 275 would be adjusted to include the nonvolatile bonding agent 220. It can be appreciated that the relative quantities of the nonvolatile components 275 and volatile components 285 and the resulting porosity of the structure 100 will vary as the material density may vary due to the reaction of the components during the reaction forming step 330. Specific examples are provided herein below.

In the mixing step 260, the fiber 210, the binder 230, the liquid 250, the pore former 240 and optionally, the bonding agent 220, if included, are mixed into a homogeneous mass of a plastically deformable and uniform mixture. The mixing step 260 may include dry mixing, wet mixing, shear mixing, and kneading, which can be necessary to evenly distribute the material into a homogeneous mass while imparting the requisite shear forces to break up and distribute or de-agglomerate the fibers 210 with the non-fiber materials. The amount of mixing, shearing, and kneading, and duration of such mixing processes depends on the selection of fibers 210 and non-fiber materials, along with the selection of the type of mixing equipment used during the mixing step 260, in order to obtain a uniform and consistent distribution of the materials within the mixture, with the desired rheological properties for forming the object in the subsequent forming step 270. Mixing can be performed using industrial mixing equipment, such as batch mixers, shear mixers, and/or kneaders. The kneading element of the mixing step 260 distributes the fiber 210 with the bonding agent 220 and the binder 230 to provide a formable batch of a homogeneous mass with the fiber being arranged in an overlapping and intertangled relationship with the remaining fiber in the batch.

The forming step 270 forms the mixture from the mixing step 260 into the object that will become the porous structure 100. The forming step 270 can include extrusion, rolling, pressure casting, or shaping into nearly any desired form in order to provide a roughly shaped object that can be cured in the reaction forming step 330 to provide the structure 100. It can be appreciated that the final dimensions of the structure 100 may be different than the formed object at the forming step 270, due to expected shrinkage of the object during the reaction forming step 330, and further machining and final shaping may be necessary to meet specified dimensional requirements. In an exemplary embodiment to provide samples for mechanical and in vitro and in vivo testing, the forming step 270 extrudes the mixture into a cylindrical rod using a piston extruder forcing the mixture through a round die.

The object is then reaction formed into the glass-ceramic composition at reaction forming step 330. Here, the object is subjected to a series of heat treatments that sequentially remove the volatile components 285 without substantially disturbing the relative position of the fiber 210, as determined by the mixing step 260, the forming step 270 and the volatile components 285 including the pore former 240, and bond the fiber into a porous construct and initiating a reaction formation of the bioactive glass fiber composition into a bioactive glass-ceramic composition to provide the porous structure 100.

The reaction forming step can include a drying phase to remove the liquid in an elevated temperature environment with or without forced convection. Various methods of heating the object can be used to dry the parts including, but not limited to heated air convection heating, vacuum freeze drying, solvent extraction, microwave or electromagnetic radio frequency (RF) drying methods. The liquid 250 within the formed object is preferably not removed so rapidly so as to contribute to cracks from shrinkage. Typically, for aqueous based systems, the formed object can be dried when exposed to temperatures between about 90° C. and 150° C. for about one hour, though the actual drying time may vary due to the size and shape of the formed object, with larger more massive objects taking longer to dry. In the case of microwave or RF energy drying, the liquid itself, and/or other components of the object, absorb the radiated energy to more evenly generate heat throughout the material During the drying phase of the reaction forming step 330, depending on the selection of materials used as the volatile components 285, the binder 230 can congeal or gel to provide greater green strength to provide rigidity and strength in the object for subsequent handling.

The reaction forming step 330 can include a binder removal to remove the binder 230 through pyrolysis, solvent extraction, and/or thermal degradation. For example, HPMC used as a binder 230 will thermally decompose at approximately 300° C. and a heat treatment phase holding a formed object at that temperature for approximately two hours can effectively remove the HPMC binder without disturbing the relative position of the fibers 210 in the formed object.

The reaction formation step 330 includes a bond formation phase that heats the formed object to a reaction and bond formation temperature resulting in the formation of fiber-to-fiber bonds at overlapping and adjacent nodes of the fiber structure. In the method of the present invention the temperature at which the reaction and bond formation temperature occurs exceeds the devitrification temperature of the glass composition of the fiber 210 so that a glass-ceramic composition is formed. The tendency of an amorphous glass composition to crystallize depends largely on glass composition, surface condition and heating and cooling rate. An amorphous glass, such as silica calcium phosphate 13-93, will precipitate wollastonite (CaSiO₃) crystals at temperatures above 800° C. when the glass is in a high surface area to mass relationship such as the fiber construct of the formed object. To ensure the homogeneous formation of wollastonite crystals in an amorphous glass matrix of the fiber structure of the present invention the bond formation phase of the reaction formation step 330 is preferably performed using a combustion of the pore former 240 to provide rapid and uniform heating throughout the volume of the formed object. The combustion of the pore former 240 in conjunction with the thermal environment during the reaction formation step 330 provides the thermal energy necessary to crystallize the fiber 210 into the desired glass-ceramic composition homogeneously throughout the internal structure of the formed object. In an embodiment of the invention greater than 50% by weight of the porous matrix is a single phase of a calcium silicate ceramic. In an alternate embodiment, greater than 40% by weight of the porous matrix is a single phase of a calcium silicate ceramic. In yet another embodiment greater than 20% by weight of the porous matrix is a single phase of a calcium silicate ceramic.

The combustion of the pore former 240 is one mechanism to provide the heating rate to initiate the formation of the glass ceramic by providing rapid and uniform heating throughout the formed object during the reaction formation step 330. The pore former 240 is preferably a combustible material such as carbon or graphite, starch, organics or polymers such as polymethyl methacrylate (PMMA), or other material that exothermically oxidizes. Generally, the pore former 240 is selected based on the temperature at which the material initiates combustion as can be determined by thermal analysis such as Thermogravimetric Analysis (TGA) or Differential Thermal Analysis (DTA) or a simultaneous combination of DTA/TGA which detects both mass loss and thermal response. For example, a DTA/TGA analysis determines the exothermic combustion point of carbon particles to be 621° C. and graphite flakes to be 603° C. that is suitable for use with a 13-93 bioactive glass composition having a devitrification temperature of approximately 800° C.

The reaction formation step 330 provides thermal environment sufficient to convert the amorphous glass composition of the fiber 210 into a glass-ceramic composition uniformly throughout the formed body through convective heat transfer from the kiln or oven in combination with a second heating source such as the exothermic combustion of the pore former 240 and holding at a temperature exceeding the devitrification temperature of the amorphous glass composition. In this way the nucleation of ceramic phase crystal structure precipitates and grows to form a glass-ceramic composition wherein the amorphous phase and the ceramic phase are bioactive and resorbable when implanted in vivo. The glass transition temperature of the amorphous glass fiber is typically exceeded during the reaction formation step 330 as the overlapping and intertangled fiber is fused into a porous rigid matrix with the distinct structural features of the fiber material becoming diminished. As the crystalline phase precipitates the amorphous glass fuses to form the porous rigid matrix. The fiber form of the bioactive glass provides preferential ordering of the crystalline phase with less grain boundary influences compared to powder-based or particle-based raw materials. Additional control over the reaction formation step 330 can be provided through controlling the heating rate of process gas in the kiln or oven to delay or accelerate the combustion of the pore former 240. For example, air or nitrogen purge flowrates can be adjusted to control the amount of oxygen present in the processing environment to support combustion or oxidation of the pore former materials. Alternative heating modes for the kiln can include microwave heating and/or direct flame heating to provide the heating rates necessary to initiate crystallization of the ceramic phase of the porous matrix.

The bonds formed between overlapping and adjacent nodes of the fibers forming the formed object can be glass bonds having a composition substantially the same as the composition of the fiber 210. The bonds can also have the same glass-ceramic composition that forms during the reaction formation step 330. The present invention provides a bioactive and resorbable tissue scaffold device that can be fabricated using medically approved materials or fabricated into medically approved materials.

Referring still to FIG. 3 an optional heat treating step 335 is performed subsequent to the reaction formation step 330 to remove internal stress in the structure and to promote crystal growth of the ceramic phase of the glass-ceramic composition. The heat treating step 335 can be performed by heating the formed object after the reaction formation step 330 to a temperature at which the glass-ceramic material will support continued growth of the ceramic phase, such as when heated above the devitrification temperature of the composition of the amorphous glass fiber. Additionally, the heat treating step 335 can relieve internal stresses that may build up during the heating/cooling cycles in the reaction formation step 330. Heat treating the glass-ceramic material of the formed object after the reaction formation step 330 involves heating the object to a temperature that is the stress relief point of the materials, i.e., a temperature at which the glass-ceramic material is hard enough to maintain its shape and form but sufficient to relieve internal stress. The heat treating temperature is determined by the composition of the glass-ceramic material. The duration of the heat treating step 335 is determined by the relative size and mass of the object. The heat treating step 335 includes a cooling phase that slowly cools the heat treated object at a rate that is limited by the heat capacity, thermal conductivity, and thermal expansion coefficient of the glass-ceramic material. Typically, most glass-ceramic compositions having a mass of approximately 10 grams can be heat treated in a kiln at a temperature that is approximately 50° C. less than the reaction formation temperature for four to six hours and cooled to room temperature over approximately four hours.

In the illustrative embodiment described above with reference to FIG. 2, a bioactive silica calcium magnesium sodium potassium phosphate fiber in the 13-93 composition is used to fabricate a test sample. 13-93 composition glass is a bioactive glass having the following composition (molar percentage): 53% SiO₂; 6% Na₂O; 10% K₂O; 5% MgO; 20% CaO; 4% P₂O₅. Fiber in this composition was drawn having a diameter of approximately 30 μm that was cut to approximately 1.2 mm length. The test sample was prepared using the method 200 by mixing 24 grams fiber, 6 grams graphite particles having an elongated shape of approximately 40 μm by 100 μm in size, 6 grams HPMC organic binder, and 15 ml deionized water. The mixture was then mixed and formed into 10 mm diameter rods and dried for two hours in a 90° C. air purged oven. The samples were then subjected to a reaction formation step by heating in a static air kiln as follows: heating to 220° C. over four hours and holding for one hour; heating to 280° C. over two hours and holding for two hours; heating to 900° C. over one hour and holding for thirty minutes; and cooling to room temperature over twelve hours. The parts were heat treated at 850° C. for four hours in a static air kiln. In the reaction formation step the calcium oxide and silica from the fiber reacted to form a single ceramic phase of wollastonite in an amorphous glass matrix. The wollastonite phase of the glass-ceramic composition can grow to fully consume the available calcium oxide resulting in a ceramic phase that is 58.57 wt % of the test sample. The composition can be calculated as shown in Table 1.

TABLE 1
Compositional Analysis

| Component | SiO₂ | CaO | MgO | Na₂O | K₂O | P₂O₅ |
|---|---|---|---|---|---|---|
| Glass Fiber (13-93) | 53.00 | 20.00 | 5.00 | 6.00 | 12.00 | 4.00 |
| Wollastonite (CaSiO₃) | 21.43 | 20.00 | | | | |
| Glass | 31.57 | 0.00 | 5.00 | 6.00 | 12.00 | 4.00 |
| Glass (wt %) | 53.91 | 0.00 | 8.54 | 10.24 | 20.49 | 6.83 |

Samples of the illustrative embodiment were tested for physical strength and bioactivity. Porosity was measured to be 52% with a standard deviation of 1%. Compressive strength was measured to be 51 MPa with a standard deviation of 14 MPa. Samples subjected to immersion in simulated body fluid exhibit the formation of hydroxyapatite consistent with materials known to demonstrate bioactivity in vitro. The single phase of wollastonite in a bioactive glass matrix exhibits physical properties sufficient to support load bearing applications as a resorbable implantable device.

The porous glass-ceramic material described herein can be used to fabricate tissue scaffolds that can be used as an implantable device for the repair and replacement of bone and bone defects. For example, tissue scaffolds of the present invention can be used in procedures such as an osteotomy (for example in the hip, knee, hand and jaw), a repair of a structural failure of a spine (for example, an intervertebral prosthesis, lamina prosthesis, sacrum prosthesis, vertebral body prosthesis and facet prosthesis), a bone defect filler, fracture revision surgery, tumor resection surgery, hip and knee prostheses, bone augmentation, dental extractions, long bone arthrodesis, ankle and foot arthrodesis, including subtalar implants, and fixation screws pins. The resorbable tissue scaffolds of the present invention can be used in the long bones, including, but not limited to, the ribs, the clavicle, the femur, tibia, and fibula of the leg, the humerus, radius, and ulna of the arm, metacarpals and metatarsals of the hands and feet, and the phalanges of the fingers and toes. The resorbable tissue scaffolds of the present invention can be used in the short bones, including, but not limited to, the carpals and tarsals, the patella, together with the other sesamoid bones. The resorbable tissue scaffolds of the present invention can be used in the other bones, including, but not limited to, the cranium, mandible, sternum, the vertebrae and the sacrum. In an embodiment, the tissue scaffolds of the present invention have high load bearing capabilities compared to conventional devices. In an embodiment, the tissue scaffolds of the present invention require less implanted material compared to conventional devices. Furthermore, the use of the tissue scaffold of the present invention requires less ancillary fixation due to the strength of the material. In this way, the surgical procedures for implanting the device are less invasive, more easily performed, and do not require subsequent surgical procedures to remove instruments and ancillary fixations.

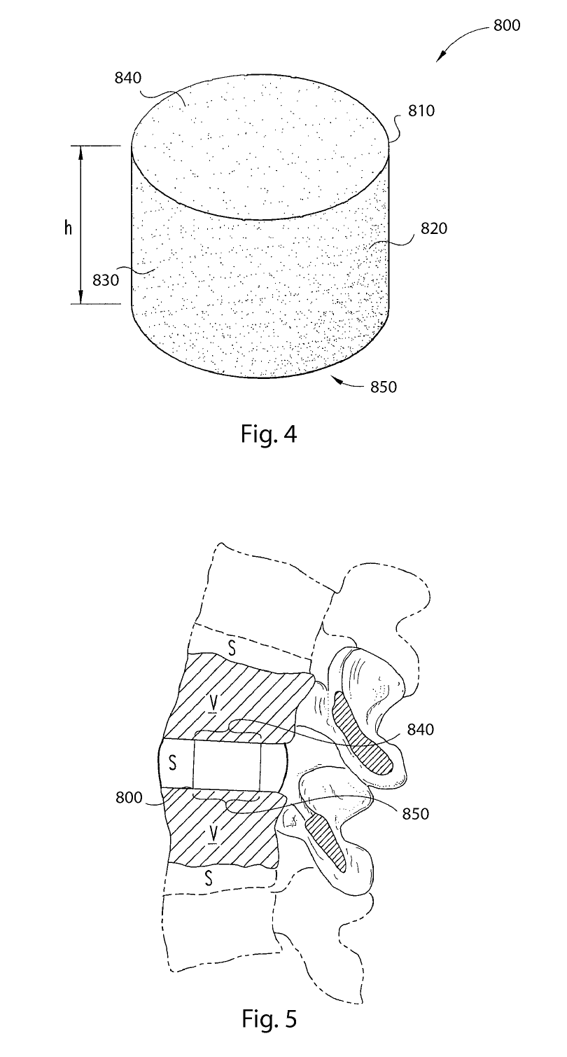

In one specific application, a tissue scaffold of the present invention, fabricated as described above, can be used as a spinal implant 800 as depicted in FIG. 4 and FIG. 5. Referring to FIG. 4 and FIG. 5, the spinal implant 800 includes a body 810 having a wall 820 sized for engagement within a space S between adjacent vertebrae V to maintain the space S. The device 800 is formed from bioactive glass-ceramic that can be formed into the desired shape using extrusion methods to form a cylindrical shape that can be cut or machined into the desired size. The wall 820 has a height h that corresponds to the height H of the space S. In one embodiment, the height h of the wall 820 is slightly larger than the height H of the intervertebral space S. The wall 820 is adjacent to and between a superior engaging surface 840 and an inferior engaging surface 850 that are configured for engaging the adjacent vertebrae V as shown in FIG. 5.

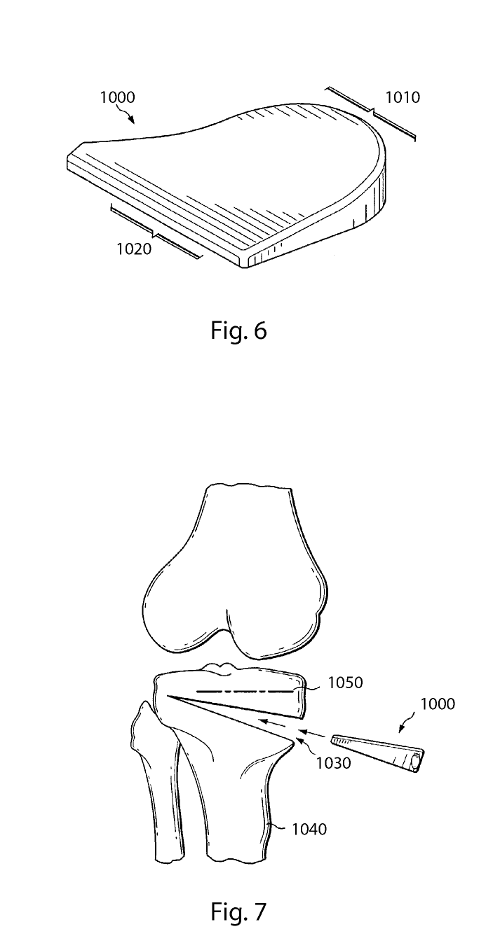

In another specific application, a tissue scaffold of the present invention, fabricated as described above, can be used as an osteotomy wedge implant 1000 as depicted in FIGS. 6 and 7. Referring to FIG. 6 and FIG. 7, the osteotomy implant 1000 may be generally described as a wedge designed to conform to an anatomical cross section of, for example, a tibia, thereby providing mechanical support to a substantial portion of a tibial surface. The osteotomy implant is formed from bioactive glass-ceramic bonded and fused into a porous material that can be formed from an extruded rectangular block, and cut or machined into the contoured wedge shape in the desired size. The proximal aspect 1010 of the implant 1000 is characterized by a curvilinear contour. The distal aspect 1020 conforms to the shape of a tibial bone in its implanted location. The thickness of the implant 1000 may vary from about five millimeters to about twenty millimeters depending on the patient size and degree of deformity. Degree of angulation between the superior and inferior surfaces of the wedge may also be varied.

FIG. 7 illustrates one method for the use of the osteotomy wedge implant 1000 for realigning an abnormally angulated knee. A transverse incision is made into a medial aspect of a tibia while leaving a lateral portion of the tibia intact and aligning the upper portion 1050 and the lower portion 1040 of the tibia at a predetermined angle to create a space 1030. The substantially wedge-shaped implant 1000 is inserted in the space 1030 to stabilize portions of the tibia as it heals into the desired position with the implant 1000 dissolving into the body as herein described. Fixation pins may be applied as necessary to stabilize the tibia as the bone regenerates and heals the site of the implant.

Generally, the use of a resorbable bone tissue scaffold of the present invention as a bone graft involves surgical procedures that are similar to the use of autograft or allograft bone grafts. The bone graft can often be performed as a single procedure if enough material is used to fill and stabilize the implant site. In an embodiment, fixation pins can be inserted into the surrounding natural bone, and/or into and through the resorbable bone tissue scaffold. The resorbable bone tissue scaffold is inserted into the site and fixed into position. The area is then closed up and after a certain healing and maturing period, the bone will regenerate and become solidly fused.

A method of filling a defect in a bone includes filling a space in the bone with a resorbable tissue scaffold comprising a glass-ceramic composition forming a porous matrix, the glass-ceramic composition of a bioactive glass phase and a wollastonite ceramic phase; and attaching the resorbable tissue scaffold to the bone.

A method of treating an osteotomy includes filling a space in the bone with a resorbable tissue scaffold comprising bioactive glass-ceramic bonded into a porous matrix, the porous matrix having a glass-ceramic composition of a bioactive glass phase and a wollastonite ceramic phase, the porous matrix having a pore size distribution to facilitate in-growth of bone tissue; and attaching the resorbable tissue scaffold to the bone.

The use of a resorbable bone tissue scaffold of the present invention as a bone defect filler involves surgical procedures that can be performed as a single procedure, or multiple procedures in stages or phases of repair. In an embodiment, a resorbable tissue scaffold of the present invention is placed at the bone defect site, and attached to the bone using fixation pins or screws. Alternatively, the resorbable tissue scaffold can be externally secured into place using braces. The area is then closed up and after a certain healing and maturing period, the bone will regenerate to repair the defect.

The present invention has been herein described in detail with respect to certain illustrative and specific embodiments thereof, and it should not be considered limited to such, as numerous modifications are possible without departing from the spirit and scope of the appended claims.

1. A porous tissue scaffold comprising:
a porous rigid matrix having a glass-ceramic composition of a bioactive glass phase and a calcium silicate ceramic phase;
the glass-ceramic composition having interconnected pore space within the porous rigid matrix to provide porosity of about 20% to about 85%; and
the interconnected pore space having a pore size sufficient to promote tissue ingrowth when implanted in living tissue.

2. The porous tissue scaffold according to claim 1 wherein the calcium silicate ceramic phase is wollastonite.

3. The porous tissue scaffold according to claim 1 wherein the calcium silicate ceramic phase is greater than 50% of the glass-ceramic composition.

4. The porous tissue scaffold according to claim 1 wherein the calcium silicate ceramic phase is greater than 40% of the glass-ceramic composition.

5. The porous tissue scaffold according to claim 1 wherein the calcium silicate ceramic phase is greater than 20% of the glass-ceramic composition.

6. The porous tissue scaffold according to claim 1 wherein the bioactive glass phase is a glass composition consisting essentially of silica, magnesium, phosphorus, sodium, and potassium.

7. The porous tissue scaffold according to claim 1 wherein the bioactive glass phase contains essentially no calcium oxide.

8. The porous tissue scaffold according to claim 1 further comprising a compressive strength of greater than 50 MPa.

9. The porous tissue scaffold according to claim 8 wherein the pore size has an average value between about 40 μm and about 600 μm.

10. The porous tissue scaffold according to claim 8 wherein the glass-ceramic composition is heat treated.

11. A method of filling a defect in a bone comprising:
filling a space in the bone with a porous tissue scaffold comprising a porous rigid matrix having a glass-ceramic composition of a bioactive glass phase and a wollastonite ceramic phase; and
inserting the porous tissue scaffold into the defect in the bone.

12. The method according to claim 11 wherein the bone is a vertebrae.

13. The method according to claim 11 wherein the porous tissue scaffold is substantially wedge-shaped and the defect is an osteotomy.
